(19) United States
(12) Patent Application Publication
(10) Pub. No.: US 2003/0026826 A1
(43) Pub. Date: Feb. 6, 2003

(54) Sugar-free chewy products and protein-based chewy products and methods for making the same

(76) Inventors: Subraman Rao Cherukuri, Vienna, VA (US); Stuart L. Cantor, Eldersburg, MD (US)

Correspondence Address: NATH & ASSOCIATES, 1030 15th STREET, 6TH FLOOR, WASHINGTON, DC, 20005, US

(21) Appl. No.: 10/024,583

(22) Filed: Dec. 21, 2001

Related U.S. Application Data
(60) Provisional application No. 60/308,566, filed on Jul. 31, 2001.

(51) Int. Cl. A61K 9/68; A61K 35/78

(52) U.S. Cl. 424/440; 424/776

(57) ABSTRACT

A sugar-free composition comprising a mixture of at least two polyols present in an amount from about 15 to 80% by weight, an emulsifier system present in an amount from about 1.0 to 30% by weight, an active agent in an amount from about 0.1 to 70% by weight and water in an amount from 0 to 15% by weight with optional components comprising colors, flavors and binders totalling 100%. Also, a method of preparing a sugar-free composition comprising the steps of mixing from about 15 to 80% by weight of at least two polyols with about 0 to 15% by weight water, heating the mixture to about 200-260° F. for about 5 to 10 minutes, cooling the mixture to about 175-240° F., adding to the mixture about 1.0 to 30% by weight an emulsifier system to form a further mixture, cooling the further mixture to about 100-110° F., and adding to the cooled further mixture about 0.1 to 70% by weight of an active ingredient to form the sugar-free composition. A protein-based composition comprising a mixture of at least one protein and one polyol in an amount of 20 to 99%, an emulsifier system in an amount of 0.0 to 30%, an active agent in an amount of 0.1 to 70%, and water in an amount of 0.0 to 10% are disclosed. Methods of making the protein-based compositions are also disclosed.

### BACKGROUND OF THE INVENTION

1. Field of the Invention

The present invention relates to a sugar-free chewy product and a protein-based chewy product and methods of preparing same. In particular, the present invention relates to a chewy product prepared from high temperature emulsions.

2. Description of the Prior Art

Pharmaceutical compositions may be produced in a variety of dosage forms, depending upon the desired route of administration of the therapeutic material. Oral dosage forms, for example, include such solid compositions as tablets, emulsions, and suspensions. The particular dosage form utilized will depend on such factors as the solubility and chemical reactivity of the pharmaceutical active. Further, the dosage form may be selected so as to optimize delivery of the pharmaceutical active and/or consumer acceptability of the composition.

Tablet compositions offer many advantages, including ease of product handling, chemical and physical stability, portability (in particular, allowing ready availability to the consumer when needed), aesthetic acceptability and dosage precision, i.e., ensuring consistent and accurate dosages of the pharmaceutical active. However, liquid formulations may offer advantages in the treatment of certain disorders, such as disorders of the upper gastrointestinal tract, wherein delivery of an active material dissolved or dispersed in a liquid ensures rapid and complete delivery to the afflicted area. In an effort to obtain the therapeutic advantages associated with liquid formulations as well as the broad advantages associated with solids, many chewable tablet formulations have been developed.

A further product often used to deliver active agents to patients are emulsions. An emulsion is a dispersed system containing at least two immiscible liquids. The majority of conventional emulsions in pharmaceutical use have dispersed particles ranging in diameter from 0.1 to 100 microns. As with suspensions, emulsions are thermodynamically unstable as a result of the excess free energy associated with the surface of the particles. The dispersed particles, therefore, strive to come together and reduce the surface area. In addition to this flocculation effect, the dispersed particles can coalesce, or fuse, and this can result in the eventual destruction of the emulsion. In order to minimize this effect, a third component, the emulsifying agent, is added to the system to improve stability. The choice of emulsifying agent is critical to the preparation of an emulsion possessing optimum stability.

Invariably, one of the two emulsified components is aqueous, while the other component is a fatty substance, such as an oil. It is unusual, but many aqueous emulsions are prepared at elevated temperatures, that is at temperatures greater than 175° F. The elevated temperature aids in the dispersal of the non-aqueous component into the aqueous component by the emulsifying agent. However, if the high temperature emulsion is cooled in order to solidify the mixture, separation of the fatty substance, or “oiling off” may observed in the final product if the levels of the fatty substance are too high or if the fatty substance is not added at the proper temperature.

A further disadvantage of high temperature emulsions is the detrimental effect of the high temperature on the efficacy and stability of active agents added to the emulsion. Many active agents, whether the active agent is a flavor, pharmaceutical, or nutriceutical, are not stable at high temperatures. Thus, if the active agents are added at the high temperatures, the active agents break down, resulting in uneven dosing (or doses that contain no active agents) and waste of the active agents.

Furthermore, many pharmaceutical and nutriceutical active agents have a bitter taste, so a flavor-enhancer or taste-masking agent is also added to oral doses containing the pharmaceutical or nutriceutical, resulting in extra care needed to ensure that neither the pharmaceutical or nutriceutical, nor the flavor enhancer is destroyed during processing of the high temperature emulsion prior to cooling to a solid base.

One way that the bitter taste of many pharmaceuticals and nutriceuticals is masked is by including the pharmaceutical or nutriceutical in a composition that includes other flavors or taste masking ingredients. Such compositions often contain sugar. For example, U.S. Pat. No. 4,582,709 discloses a chewable mineral supplement and the process for making the same. The supplement contains the mineral compound in a soft, nougat candy base. The base is comprised of about 20-40% by weight sugars and about 13-41% by weight corn syrup.

U.S. Pat. No. 4,778,676 discloses a chewable delivery system for drugs such as cholestyramine and fibers comprising a water-insoluble pre-coated active which is taste masked and a confectionary matrix which comprises a binder system comprising gelatin and a humectant material. Flavors and artificial sweeteners are added to the confectionary matrix. A method of preparing the confectionary delivery system is also disclosed.

U.S. Pat. No. 5,637,313 discloses a soft, chewable dosage form, including a matrix of hydrogenated starch hydrolysate, a water soluble bulking agent, and a water insoluble bulking agent. A method of preparing the soft, chewable dosage form is also disclosed.

One disadvantage of including the pharmaceuticals and nutriceuticals in a composition which contains sugars is the caloric content of the sugars. The high calorie content of the compositions containing sugar makes the compositions unattractive to those people trying to reduce their weight.

Further, the sugars in the compositions help to promote the growth of bacteria in the mouth of the person chewing the composition. The growth of bacteria is one of the leading causes of tooth decay among humans. Thus, the use of sugars in compositions containing pharmaceuticals and nutriceuticals has been held to a minimum.

Some of the disadvantages associated with compositions containing sugars have been addressed by developing compositions that are sugar-free. For example, Canadian Patent Application No. 2,165,838 discloses sugar-free hard boiled candies. The hard sugar-free candies are prepared by boiling a mixture of polyols dissolved in water. The sugar-free compositions displayed non-sticky characteristics, and did not deform in high ambient temperatures.

Likewise, U.S. Pat. No. 4,971,798 discloses a hard composition containing a medicinally active ingredient. The hard, sugar-free composition is prepared with hydrogenated isomaltulose in place of the sugar. The medicinally active ingredients are selected from antitussives, decongestants, antihistamines and expectorants.

A further sugar-free composition is disclosed in U.S. Pat. No. 4,963,359. The '359 patent discloses a composition free of cellulosics and graining compounds containing hydrogenated starch hydrolysates, at least one edible oil, fat, wax or mixtures thereof and an emulsifying agent. The composition is a mixture of a viscous liquid matrix and a semi-solid matrix. The compositions provide a range of hardness from hard candies to soft chew candies.

As can be seen by the '359 patent above, soft sugar-free compositions have also been prepared. Another such soft sugar-free composition is disclosed in European Patent No. 0 273 001. The '001 patent discloses soft, sugarless aerated confectionery compositions. The compositions are prepared from hydrogenated starch hydrolysates mixed together with water soluble and water insoluble cellulosics. The '001 patent also discloses a method for preparing such soft, sugarless aerated confectionery compositions.

U.S. Pat. No. 5,637,313 discloses a soft, chewable dosage form comprising a matrix of hydrogenated starch hydrolysate, a water soluble bulking agent, and a water insoluble bulking agent. Antacids are the preferred insoluble bulking agent. The patent also contemplates the incorporation of active materials into the matrix.

As can be seen above, it is known to include active ingredients in sugared and sugar-free compositions. An active ingredient that is often difficult to incorporate into such compositions is fiber. U.S. Pat. No. 5,476,678 discloses a composition for and method of producing a fiber fortified chewy confectionery. The confection has a fructose base, as well as supplemental dietary fiber of up to 7 grams per ounce of confection. The confection is in the form of nougat or taffy that is chewy in nature.

Likewise, U.S. Pat. No. 4,778,676 discloses a sugarless confectionery delivery system for drugs and fiber. The sugarless confectionery system includes a coated water insolubilized active and a confectionery matrix comprising a binder system of gelatin and a humectant.

However, there is still a need for a sugar-free chewy composition prepared from a high temperature emulsion in which the composition is reduced in caloric value, thus being ideal for people trying to reduce their weight. In addition, there is a need for a chewy composition which does not promote tooth decay, and provides good mouth-feel for the person chewing the product. Also, there is a need for a sugar-free chewy product that delivers an active ingredient, including fiber, to the individual chewing the composition.

It has been found that the present inventive subject matter provides a sugar-free way of delivery of active ingredients to a mammal without promoting tooth decay or adding much caloric intake to the user. The inventive subject matter is especially conducive for aiding in the weight loss program of the user, as well as delivering the needed active ingredients, including fiber. Further, the active agents present in the sugar-free chewy compositions are not deteriorated or destroyed by the high temperatures used in preparing compositions. Other objects and advantages of the present inventive subject matter are expressed herein.

### BRIEF SUMMARY OF THE INVENTION

Applicant has unexpectedly developed a novel sugar-free composition comprising:

a) a mixture of at least two polyols present in an amount from about 15 to 80% by weight;

b) an emulsifier system present in an amount from about 1.0 to 30% by weight;

c) an active agent in an amount from about 0.1 to 70% by weight; and

d) water in an amount from 0 to 15% by weight; and

e) optional components comprising colors, flavors and binders, wherein said ingredients in said sugar-free composition are present in an amount totaling 100%.

Applicant has further developed a method of preparing a sugar-free composition comprising the steps of:

a) mixing from about 50 to 99% by weight of at least two polyols with about 0 to 25% by weight water;

b) heating said mixture to about 200-260° F. for about 5 to 10 minutes;

c) cooling said mixture to about 175-240° F.;

d) adding to said mixture about 1.0 to 30% by weight an emulsifier system to form a further mixture;

e) cooling said further mixture to about 100-110° F.;

f) adding to said cooled further mixture about 0.0 to 20% by weight of an active ingredient to form said sugar-free composition.

Further, Applicant has unexpectedly developed an oral hygiene composition comprising:

a) a mixture of at least two polyols present in an amount from about 15 to 80% by weight;

b) an emulsifier system present in an amount from about 1.0 to 30% by weight;

c) an active ingredient in an amount from about 0.0 to 20% by weight;

d) a bioadhesive agent for improving oral hygiene; and

e) water in an amount from 0 to 15% by weight.

Still further, Applicant has unexpectedly developed a weight reduction or cholesterol reducing composition comprising:

a) a mixture of at least two polyols present in an amount from about 15 to 80% by weight;

b) an emulsifier system present in an amount from about 1.0 to 30% by weight;

c) an active agent in an amount from about 0.1 to 70% by weight, said active agent being selected from the group consisting of dietary fibers, hydroxycitric acid, stannol esters, vitamins, minerals, and mixtures thereof; and

d) water in an amount from 0 to 15% by weight.

Applicant has also unexpectedly developed a protein-based, sugar-free composition comprising:

a) a mixture of at least one protein and at least one polyol present in an amount from about 20 to 99% by weight;

b) an emulsifying system present in an amount from about 1.0 to 30% by weight;

c) an active agent in an amount from about 0.1 to 70% by weight; and

d) water in an amount from 0 to 15% by weight.

### DETAILED DESCRIPTION OF THE EMBODIMENTS

The present inventive subject matter is a sugar-free composition which includes a mixture of at least two polyols present in an amount from about 15 to 80% by weight, an emulsifier system present in an amount from about 1.0 to 30% by weight, an active agent in an amount from about 0.1 to 70% by weight and water in an amount from 0 to 15% by weight. The composition may optionally contain colors, flavors and binders, such that the combination of ingredients in the sugar-free composition are present in an amount totaling 100%.

The present inventive subject matter is also drawn to an oral hygiene composition including a mixture of at least two polyols present in an amount from about 50 to 99% by weight, an emulsifier system present in an amount from about 1.0 to 30% by weight, an active ingredient in an amount from about 0.0 to 20% by weight, a bioadhesive agent for improving oral hygiene and water in an amount from 0 to 15% by weight. Still further, Applicant has unexpectedly developed a weight reduction composition including a mixture of at least two polyols present in an amount from about 15 to 80% by weight, an emulsifier system present in an amount from about 1.0 to 30% by weight, an active agent in an amount from about 0.1 to 70% by weight, said active agent being selected from the group consisting of dietary fiber, stannol esters, vitamins, minerals, and mixtures thereof, and water in an amount from 0 to 15% by weight. The compositions are generally soft chew in nature, meaning that the compositions are not hard candies, nor are the compositions liquid. The present inventive subject matter also contemplates the method of making the sugar-free compositions.

As used herein, the terms “sugar-free” and “sugarless” refer to the compositions that are substantially free (i.e. contain less than 1% by weight, and preferably less than 0.1% by weight) of fermentable sugars such as glucose and corn syrups, which help promote tooth decay. The terms “sugar-free” and “sugarless” are meant to be used interchangeably within this application.

As used herein, the expression “mammal” includes without limitation any mammalian subject, such as mice, rats, guinea pigs, cats, dogs, human beings, cows, horses, sheep or other livestock.

The expression “soft chew” includes items which are solid at room temperature, and those items which may be considered semi-solid or soft chew at room temperature. In general, the term “soft chew” is meant to cover those items that are not liquid or gas at room temperature.

The expression “emulsifier system” means a component that comprises emulsifiers, fats and mixtures thereof.

As used herein, the expression “active agent” includes, without limitation, dietary fiber, stannol esters, therapeutically active substances, vitamins, minerals, antacids, cough and cold medications, analgesics, cardiovasular medications, anti-smoking, psycho-therapeutics, antibiotics, and mixtures thereof.

The unique sugar-free compositions of the present inventive subject matter are useful for many mammalian needs. One such use of the sugar-free compositions of the present inventive subject matter are to help promote good oral hygiene. Good oral hygiene is imperative for keeping the mouth free from diseases of the teeth and gums and defects in the teeth. In particular, good oral hygiene helps prevent the onset of tooth decay in the form of cavities, discolorations of the teeth, malformations of the teeth, loss of teeth, formation of plaque, inflammation of the dental pulp, gingivitis (swelling or bleeding of the gums), and inflammation of the periodontium.

The present inventive compositions help promote good oral hygiene because the compositions are composed of ingredients that do not promote the growth of bacteria in the mouth. It is often the growth of bacteria that leads to the onset of the above problems within the mouth. Fermentable sugars such as glucose and corn syrup contain ingredients that help promote the growth of bacteria in the mouth. Thus, since the present inventive compositions are sugar-free, the compositions do not aid in the growth of bacteria, and accordingly do not promote the onset of the above oral problems.

In addition, the compositions of the present inventive subject matter exhibit good bioadhesive properties. The good bioadhesive properties help prolong the positive aspects of the compositions in the mouth of the user. Furthermore, the compositions of the present inventive subject matter provide good sublingual delivery of active ingredients in the mouth A further use of the sugar-free compositions of the present inventive subject matter is as a product to be used by those individuals trying to lose weight. Weight reduction is important in today's society, as more and more people think that they are overweight, and thus would like to lose a few pounds. One problem for those trying to lose weight is to find a suitable snack or treat that will aid in the reduction of weight.

A strict regimen is often needed if weight loss is to be attained in an individual. The regimen sets forth when, what and how much food an individual may intake during a given day. Many times, the individual desires a small portion of food between the scheduled meals. In order to maintain the regimen, the person must snack on low caloric foods between the scheduled meals. The present inventive compositions would be ideal as such a snack, since the sugar-free compositions of the present inventive subject matter are light in caloric value. The lack of fermentable sugars and corn syrups greatly reduces the dental cariodenicity of the compositions.

In addition, an individual trying to reduce their weight must still maintain an input of nutrients. As is further discussed below, the active agents included in the present inventive subject matter include necessary vitamins, minerals, dietary fiber, and other nutrients essential to a persons well-being. The incorporation of those active agents into the sugar-free compositions creates an ideal delivery system of those active agents to a person trying to lose weight.

An even further ideal use for the sugar-free compositions of the present inventive subject matter are as food snacks for people suffering from diabetes. In general terms, diabetes is a syndrome characterized by hyperglycemia resulting from an absolute or relative impairment in insulin secretion and/or insulin activity. The most common type of diabetes, referred to as Type II diabetes, is treated with a controlled diet, exercise and oral drugs. Type II diabetes is a heterogeneous group of disorders in which hyperglycemia results from both an impaired insulin secretory response to glucose and decreased insulin effectiveness in stimulating glucose uptake by skeletal muscle.

As can be seen, type II diabetics have difficulty processing glucose. Thus, many foods that include glucose and other sugars cannot be eaten by persons with diabetes. The compositions of the present inventive subject matter are made without glucose or other fermentable sugars, and therefore are ideal as snack foods for people suffering from diabetes.

The first component of the sugar-free compositions of the present inventive subject matter is a mixture of at least two polyols. The mixture of polyols is present in the inventive compositions in an amount from about 15 to about 80% by weight of the final weight of the composition. Preferably, the mixture of polyols is present in an amount of about 20 to 50% by weight. Polyols useful in the present inventive compositions include, without limitation, hydrogenated starch hydrolysate, isomalt, erythritol, polydextrose, maltitol, lactitol, glycerin, sorbitol, xylitol, mannitol and mixtures thereof. It has been found that the polyols act synergistically together to produce a base which is flexible and non-staling over time. Also, the synergistic effect produces a base that does not stick to teeth or packaging materials. Further, the compositions of the inventive subject matter have low sorbitol contents (generally less than 2%), which also aids in the non-stick properties of the compositions.

The second component of the sugar-free compositions of the present inventive process comprises about 1.0 to 30.0% by weight of an emulsifier system. The emulsifier system comprises emulsifiers, fats and mixtures thereof. Generally, the emulsifier system comprises 0.5 to 20.0% (by weight of the final composition) of at least one emulsifier and 1.0 to 10.0% by weight (also of the final composition) of at least one fat.

The function of the emulsifier is to prevent the oil or fat phase from separating from the polyols of the product. The emulsifier also provides good aeration in the buccal cavity during chewing. Emulsifiers work well to provide a smooth mouthfeel and help prevent the product from sticking to the packaging materials. However, there is a critical level of usage for the emulsifier beyond which the beneficial effect of the emulsifier will be negated. In other words, too high a percentage of emulsifiers will result in a reduction of the bite force of the stable solid delivery system and act to create a softer product.

Examples of emulsifiers that work well in the present inventive process include, without limitation, acetylated monoglycerides, glycerol esters, lecithin, de-oiled lecithin, enzyme-modified lecithins, purified lecithins, glycerol monostearate, polyglycerol esters, propylene glycol esters, sorbitan esters, polysorbate esters, sodium laurel sulfate, polyethylene glycols, sorbitol mono-, di- and tri-stearates and mixtures thereof. Selection of the proper emulsifier will depend on the desired characteristics of the final sugar-free composition.

The emulsifier system also includes at least one fat component. The fats are chosen based on their solid fat index (SFI), active oxygen stability and melting characteristics at mammalian body temperature. The fat component of the emulsifier system acts to improve the pliability of the final sugar-free composition. However, if the level of fats in the composition is too high, or the fats are added at too high of a temperature, separation or “oiling off” is observed during handling of the compositions. By adding the emulsifier system at the correct temperature, the oil-soluble compounds become embedded in the polyols, and form an oil-in-water emulsion.

Examples of fats that may be used in the emulsifier system include, without limitation, chocolate, cocoa butter, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate, and mixtures thereof. Selection of the fat component for use in the emulsifier system will depend on the desired characteristics of the final stable solid delivery system.

The sugar-free compositions of the present inventive subject matter may also include water in an amount from about 0 to 15% by weight of the final sugar-free composition.

In a preferred embodiment of the present inventive subject matter, the sugar-free compositions also include a viscosity improvement agent. The viscosity improvement agents help improve the viscosity of the polyol/water mixture. An improved viscosity is important in providing a final sugar-free composition that is chewy in nature. Generally, a reduction in moisture increases the viscosity, which in turn results in a chewier product. However, a viscosity improvement agent provides the same properties without having to reduce the water content in the composition.

The viscosity improvement agent is present in amounts from about 0.1 to 10% by weight of the final composition. The viscosity improvement agent may include, without limitation, locust bean gum, guar gum, hydrolyzed guar gum (benefiber) carrageenan, starches, gum arabic, gelatin, agar, alginate, pectin and mixtures thereof.

In another preferred embodiment of the present inventive subject matter, the sugar-free compositions further comprise a bioadhesive agent. The bioadhesive agent helps the sugar-free compositions be retained in the mouth during chewing, thus aiding in improving the oral hygiene of the user.

Bioadhesive agents useful in the present inventive subject matter include, without limitation, hydroxypropylmethyl cellulose, ethyl cellulose, acrylic esters, polyvinyl acetates, alcohols and gums.

In all of the above embodiments of the present inventive subject matter, at least one active agent is mixed with the stable solid delivery system. In general, the active ingredient is present in the inventive compositions in an amount from about 0.1 to about 70% by weight of the final composition. Preferably, the active ingredient is present in an amount from 1.0 to 50% by weight. One such preferred active ingredient is dietary fiber. The term “dietary fiber” is understood to mean the component of food which is non-digestible and non-metabolizible by humans. It is well known, however, that dietary fibers as they occur naturally in food sources also have associated with them a small digestible portion comprising fats, proteins, and carbohydrates.

Dietary fiber can be divided into two broad categories: insoluble dietary fiber and water soluble dietary fiber. For purposes of this invention, “insoluble dietary fiber” means the water insoluble portion of an edible material remaining after chemical and enzymatic treatment has removed proteins, fats and carbohydrates. For example, brans, celluloses, hemicelluloses lignin and the like, are among those useful. “Soluble dietary fiber” means dietary fiber which is the water soluble portion of an edible material remaining after the chemical and enzymatic treatment has removed proteins, fats and carbohydrates. For example, pectin, guar gum, locust bean gum, gum arabic, karaya gum and others from the galacturonan and galactomannan classes; as well as psyllium seed gum, carageenan, konjac mannan, among others. These soluble fibers have been known to inhibit absorption of cholesterol in mammals, as well as re-absorption of bile salts. The mechanism for this benefit is believed to be three-fold in nature. First, the shear mass of the swelled fiber occludes the cholesterol and bile salts, thereby preventing absorption. Second, the fibers will absorb the cholesterol and bile salts, thereby physically transporting them out of the body. Finally, the fibers increase the transit time of stool bulk, which decreases the time in which absorption of cholesterol and bile salts can occur. Dietary fiber provides the bulking effect commonly associated with fiberous materials.

Useful dietary fiber substrates include noncellulosic polysaccharides, pectin, gums, algal polysaccharides, recently developed specialty maltodextrins, cellulose, hemicellulose, fructo-oligo saccharides, psyllium, lignin, mucilages and mixtures thereof. The dietary fiber is present in the compositions in amounts of about 0.1% to about 20% by weight.

Further, the active agent may be selected from the group consisting of therapeutically active substances, stannol esters, vitamins, minerals, antacids, cough and cold medications, analgesics, cardiovasular medications, anti-smoking, psycho-therapeutics, antibiotics, and mixtures thereof.

Examples of therapeutically active substances that may be active agents in the present inventive process include, without limitation, antitussives, antihistamines, decongestants, alkaloids, mineral supplements, laxatives, vitamins, antacids, ion exchange resins, anti-cholesterolemics, antiarrhythmics, antipyretics, analgesics including acetaminophen, aspirin, non-asteroidal anti-inflammatory drugs (“NSAID”) and opioids, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, anti-diarrheal preparations, anti-anginal drugs, vasodilators, anti-hypertensive drugs, vasoconstrictors, migraine treatments, antibiotics, tranquilizers, anti-psychotics, antitumor drugs, anticoagulants, antithrombotic drugs, hypnotics, anti-emetics, anti-nausants, anti-convulsants, neuromuscular drugs, hyper-and hypoglycemic spasmodics, uterine relaxants, mineral and nutritional additives, antiobesity drugs, anabolic drugs, erythropoetic drugs, antiasthmatics, cough suppressants, mucolytics, anti-uricemic drugs and mixtures thereof.

Further preferred nutritional active materials useful in the present inventive subject matter include, without limitation, calcium-containing materials such as calcium carbonate, stannol esters, hydroxycitric acid, vitamins, minerals, herbals, spices and mixtures thereof.

Examples of vitamins that are available as active ingredients include, without limitation, vitamin A (retinol), vitamin D (cholecalciferol), vitamin E group (α-tocopherol and other tocopherols), vitamin K group (phylloquinones and menaquinones), thiamine (vitamin B₁), riboflavin (vitamin B₂), niacin, vitamin B₆ group, folic acid, vitamin B₁₂ (cobalamins), biotin, vitamin C (ascorbic acid), and mixtures thereof. The amount of vitamin or vitamins present in the final encapsulated product of the present inventive subject matter is dependent on the particular vitamin and is generally the United States' Department of Agriculture Recommended Daily Allowances (USRDA) for that vitamin. For example, if vitamin C is the active ingredient and the encapsulated product is being used in a confectionery or chewing gum targeting adults, the amount of vitamin C in the encapsulated product would be 60 milligrams, which is the USRDA of vitamin C for adults.

Examples of minerals that are available as active ingredients include, without limitation, calcium, magnesium, phosphorus, iron, zinc, iodine, selenium, potassium, copper, manganese, molybdenum and mixtures thereof. As is the case with vitamins, the amount of mineral or minerals present in the final encapsulated product of the present inventive subject matter is dependent on the particular mineral and is generally the USRDA for that mineral. For example, if iodine is the active ingredient and the encapsulated product is being used in a confectionery or chewing gum targeting adults, the amount of iodine in the encapsulated product would be 150 micrograms, which is the USRDA of iodine for adults.

The sugar-free compositions of the present inventive subject matter also contemplate adding additional ingredients along with the active agent. The additional ingredients are selected from the group consisting of colors, flavors, sweeteners, surfactants, preservatives, bulking agents, and mixtures thereof.

Flavors may be chosen from natural and synthetic flavor liquids. Flavors useful in the present inventive process include, without limitation, chocolate, volatile oils, synthetic flavor oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral, i.e., alphacitral (lemon, lime), neral, i.e., betal-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), and mixtures thereof.

In a preferred embodiment, a lemon-lime flavor for healthy chews comprises total liquid and powder flavors in an amount of 3.5-4.0% by weight. Such flavors are refreshing, mouthwatering and cause salivation. Such flavors include caramel, vanilla and derivatives such as vanillan, lemon-lime liquid flavor, lemon-lime flavor encapsulation, freeze-dried orange crystals, and cream powder flavor.

In another preferred embodiment, a tropical flavor for healthy chews comprises a total tropical liquid and powdered flavor form in an amount of 3.6-4.1% by weight. Such a flavor is refreshing, mouthwatering and causes salivation.

In yet another preferred embodiment, an oral hygiene composition for healthy chews comprises total liquid and powder flavors in an amount of 2.9-3.5% by weight. Such flavors include cool-mint flavor, menthol flavor encapsulation, eucalyptus oil, peppermint liquid, and peppermint flavor encapsulation.

Further examples of flavors useful in the inventive process include, without limitation, beef flavorings, chicken flavorings, rice flavorings, lamb flavorings, pork flavorings, seafood flavorings, and mixtures thereof.

The sweeteners may be chosen from the following non-limiting list: saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, zylitol, and the like. Also contemplated are hydrogenated starch hydrolysates and synthetic sweetener 3,6-dihydro-6-methyl-1-1-1,2,3-oxathiazin-4-one-2,2-dioxide, particularly the potassium salt (acesulfame-K) and sodium and calcium salts thereof. Other sweeteners may also be used.

A preferred embodiment of the present inventive subject matter is drawn to an oral hygiene composition comprising a mixture of at least two polyols present in an amount from about 15 to 80% by weight, an emulsifier system present in an amount from about 1.0 to 30% by weight, an active ingredient in an amount from about 0.1 to 70% by weight, a bioadhesive agent for improving oral hygiene and water in an amount from 0.0 to 15% by weight. Each component is as defined above and given the same meaning as above.

A still further embodiment of the present inventive subject matter is drawn to a weight reduction composition comprising a mixture of at least two polyols present in an amount from about 15 to 80% by weight, an emulsifier system present in an amount from about 1.0 to 30% by weight, an active agent in an amount from about 0.1 to 70% by weight, said active agent being selected from the group consisting of dietary fiber, vitamins, minerals, and mixtures thereof and water in an amount from 0.0 to 25% by weight. Again, each component is as defined above and given the same meaning as above.

The present inventive subject matter is also drawn to the method of making the above sugar-free compositions. The first step in the method of the present inventive subject matter comprises mixing from about 15 to 80% by weight of at least two polyols, as defined above, with water present in an amount of about 0.0 to 15% by weight. The polyols are mixed in a conventional mixer or cooker. The mixer or cooker may be a batch or continuous process mixer or cooker. The mixture is then heated to a temperature of about 200-260° F. This temperature is maintained for about 5 to 10 minutes, allowing some of the moisture to be evaporated off. When the moisture level is about 12% by weight water, or a Brix of about 87 is attained, the mixture is cooled to a temperature of about 175-240° F.

To the cooled mixture is added about 1.0 to 30% by weight of an emulsifier system. Again, the emulsifier system is identical to the emulsifier system define above with respect to the compositions of the present inventive subject matter. The addition of the emulsifier system to the cooled mixture forms a further mixture. The further mixture is then cooled, and the above active agents, in amounts of up to 0.1 to 70% by weight are added, thus forming the sugar-free composition. Other ingredients, such as flavors, colors, sweeteners, bulking agents, and the like may also be added at this time. The final mixture is then formed into the final product and allowed to set up.

In a preferred embodiment of this invention, a viscosity improvement agent is added to the polyol/water mixture in the first step of the process. The viscosity improvement agent is added in amount of about 0.1 to 10% by weight. The viscosity improvement agent is as defined above.

In still another preferred embodiment of this invention, a bioadhesive agent is added with the active agent in the last step of the inventive method. The bioadhesive agent is as defined above.

In a yet further embodiment of the present inventive subject matter, the inventive compounds may be made by the following alternative method. The emulsifiers in the composition may be melted independent of the polyols or proteins. Generally, the emulsifiers will be melted at a temperature of about 110° F. The melted emulsifiers will then be mixed with other components including the active agents, colors, sweeteners, flavors, etc., at a relatively constant temperature until homogeneous. Upon the mixture becoming homogeneous, the polyols and/or proteins are then added to the mixture to form a further mixture. The further mixture is mixed until homogeneous, after which the product is formed and allowed to set up.

In yet a still further embodiment of the method of the present inventive subject matter, the heating of the mixture in the second step may be done under vacuum, thus allowing for a shorter time needed for mixing the polyols with the water.

The incorporation of the active agents after the sugar-free composition has been formed provides great flexibility for the uses of the present inventive process. For example, the active agents may be incorporated into the sugar-free compositions immediately after cooling the composition to a temperature around 110° F., thus resulting in a final product which may be formed and packaged in conventional manners for shipment and delivery to a customer. In this example, the active agents and sugar-free composition are mixed in the same plant or facility in which the sugar-free composition was prepared. Also, the preparation of the sugar-free composition and the mixing of the active agent are done within a short period of time (within a couple of hours) of each other.

Another advantage of the sugar-free composition is that the active agents may be incorporated thereinto at a later time. For example, the sugar-free composition may be prepared by the inventive process and intermediately packaged for storage. Then at a later date (days, weeks or months after initial preparation of the delivery system), the sugar-free composition is reheated and the active agents are incorporated into the sugar-free composition, resulting in a final product that is formed and packaged by conventional means and delivered to the customer. It is contemplated within the scope of the present inventive process that this later incorporation of the active agents into the sugar-free composition may take place at the same site as did the preparation of the sugar-free composition, or it may take place at a remote location.

As used herein, “remote location” means a location that is separate from the location in which the sugar-free composition is prepared according to the present inventive process. The remote location may be another building within the same complex as the facility in which the delivery system was prepared, or it may be at a location that is away from the delivery system production site. Of course, if the remote location is a site that is away from the production site, then the intermediately packaged sugar-free composition must be transported to the remote location. The present inventive subject matter contemplates all modes of transporting the sugar-free composition to the remote location, including without limitation, by truck or other automotive vehicle, airplane, train, or ship.

In a preferred embodiment of the present inventive process, the sugar-free composition is formed into a desired shape and cooled to room temperature. After a period of time, the sugar-free composition is reheated to a temperature of about 110° F. and at least one active agent is mixed therewith to form the final product. The reheating of the sugar-free composition may take place at a remote location, as is discussed above.

In another preferred embodiment of the present inventive process, the sugar-free composition is formed into a desired shape and cooled to room temperature, after which the sugar-free composition is packaged for transport to a remote location. After the sugar-free composition is transported to the remote location, it is removed from the packaging and heated to a temperature of about 110° F. and mixed with at least one therapeutically active substance to form a pharmaceutical composition.

Another aspect of the inventive subject matter, is a protein-based sugar-free composition embodiments of which comprise: a) a mixture of at least one protein and at least one polyol present in an amount from about 20 to 99% by weight; b) an emulsifier system present in an amount from about 1.0 to 30% by weight; c) an active agent in an amount from about 0.1 to 70% by weight and d) water in an amount from about 0.0 to 10% by weight.

Polyols used in the above-described protein-based compositions include but are not limited to hydrogenated starch hydrolysate, isomalt, erythritol, polydextrose, maltitol, lactitol, glycerin, sorbitol, xylitol, mannitol and mixtures thereof. The emulsifier system, as in the sugar-free composition, contains at least one emulsifier and at least one fat with the emulsifier being present in an amount of about 0.5 to 20% by weight and the fat is present in an amount of about 1.0 to 10% by weight of the final composition.

The fat component of the emulsifier system of the protein-based compositions can include but is not limited to any one of the following: chocolate, cocoa butter, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate, and mixtures thereof. The emulsifier can include but is not limited to any one of the following: acetylated monoglycerides, glycerol esters, lecithin, de-oiled lecithin, enzyme-modified lecithins, purified lecithins, glycerol monostearate, polyglycerol esters, propylene glycol esters, sorbitan esters, polysorbate esters, sodium laurel sulfate, polyethylene glycols, sorbitol mono-, di- and tri-stearates and mixtures thereof.

Actives used in the protein-based, sugar free compositions can include but are not limited to the following: dietary fiber, therapeutically active substances, vitamins, minerals, antacids, cough and cold medications, analgesics, cardiovascular medications, anti-smoking, psycho-therapeutics, antibiotics, and mixtures thereof.

A viscosity improvement agent may be used and is present in an amount of 0.1 to 10% by weight of the final composition. Examples of such agents include locust bean gum, guar gum, hydrolyzed guar gum (benefiber) carrageenan, starches, gum arabic, gelatin, agar, alginate, pectin and mixtures thereof. Bioadhesive agents, such as hydroxypropylmethyl cellulose, ethyl cellulose, acrylic esters, polyvinyl acetates, alcohols, and gums may also be used in the protein-based compositions.

The polyol of the embodiments of the protein-based compositions herein described function to soften the protein with the polyol thus expanding the plasticizing of the protein and allowing the composition to stretch. The polyol allows the protein to expand three times its size and enables the use of more protein and enhances the chew characteristics of the protein-based compositions. The prior art compositions, using water and protein, conversely, expanded to 50 times its size; however, the skilled practitioner is forced to use less protein.

Also contemplated for use in the protein base of this composition includes gums such as alginates, xanthan gum, guar gum, hydrolyzed guar gum, other gums, polysaccharides, and agar. Other proteins useful in the above described protein base include casein, milk proteins, and fish proteins.

In a preferred embodiment of the protein-based, sugar-free composition, the at least one protein is present in an amount of from about 1 to 35% by weight.

In yet another preferred embodiment, of the protein-based, sugar-free composition, the polyol is present in an amount of from about 1.5 to 70% by weight.

In still another preferred embodiment, of the protein-based, sugar-free composition, the polyol is present in an amount of from about 1.5 to 2.0 times the amount of the protein.

It will also be understood by those of skill in the art that the protein-based compositions containing other actives are also possible and within the spirit of the invention. Such actives could include pharmaceuticals and nutriceuticals.

Examples of pharmaceuticals include, but are not limited to, OTC medications, antacids, cough and cold medications, and laxatives. Non-limiting examples of nutriceuticals include calcium, glucosamine and fiber.

The present inventive subject matter is also drawn to the method of making the above protein-based, sugar-free compositions. The first step in the method of the present inventive subject matter comprises mixing from about 15 to 80% by weight at least one protein and a polyol, as defined above, with water present in an amount of about 0.0 to 10%. The protein-polyol mixture is mixed in a conventional mixer or cooker. The mixer or cooker may be a batch or continuous process mixer or cooker. The mixture is then heated to temperature of about 200-260° F. This temperature is maintained for about 5 to 10 minutes, allowing some of the moisture to be evaporated off. When the moisture level is about 12% by weight water, or a Brix of about 87 is attained, the mixture is cooled to a temperature of about 175-240° F.

To the cooled mixture is added about 1.0 to 30% by weight of an emulsifier system. Again, the emulsifier system is identical to the emulsifier system defined above with respect to the compositions of the present inventive subject matter. The addition of the emulsifier system to the cooled mixture forms a further mixture. The further mixture is then cooled, and the above active agents, in amounts of up to 0.1 to 70% by weight are added, thus forming the protein-based, sugar-free composition. Other ingredients, such as flavors, colors, sweeteners, bulking agents, and the like may also be added at this time. The final mixture is then formed into the final product and allowed to set up.

The following examples are given to illustrate the invention, but are not deemed to be limiting thereof. All percentages given throughout the specification are based upon weight unless otherwise indicated.

### EXAMPLES

### Example 1

### Preparation of Sugar-Free Composition#1

A sugar-free base was prepared from 55% maltitol syrup (containing less than 2% sorbitol) and 20% sorbitol, 0.55% kappa carrageenan and 20% water being added to a heating vessel. Following mixing, the mixture was heated to about 240° F. and a Brix of 87 to form a cooked mixture.

The cooked mixture was cooled to about 185-235° F. Next, 8% partially hydrogenated soy and cottonseed oil, 1.4% lecithin, and 2.5% mono and diglycerides, were then mixed. The fats were mixed for two minutes until homogenous. The fats were then mixed with 75% of the above cooled cooked mixture. The mixture was allowed to cool, forming a sugar-free composition.

The sugar-free composition was formed and allowed to setup.

### Example 2

### Preparation of Sugar-Free Composition #2

A sugar-free base was prepared from 55% maltitol syrup and 20% lactitol, 0.55% kappa carrageenan and 20% water being added to a heating vessel. Following mixing, the mixture was heated to about 240° F. and a Brix of 87 to form a cooked mixture.

The cooked mixture was cooled to about 185-235° F. Next, 5.5% partially hydrogenated soy and cottonseed oil, 1.4% lecithin, 2.5% mono and diglycerides, and 3.0% hydrogenated soy oil were then mixed. The fats were mixed for two minutes until homogenous. The fats were then mixed with 75% of the above cooled cooked mixture. The mixture was allowed to cool, forming a sugar-free composition.

The sugar-free composition was formed and allowed to set-up.

### Example 3

### Preparation of Sugar-Free Composition #3

A sugar-free base was prepared from 55% hydrolyzed starch hydrogenate (HSH) and 20% lactitol, 0.55% iota carrageenan and 20% water being added to a heating vessel. Following mixing, the mixture was heated to about 240° F. and a Brix of 87 to form a cooked mixture.

The cooked mixture was cooled to about 185-235° F. Next, 8.0% partially hydrogenated soy and cottonseed oil, 1.4% lecithin, and 4.5% mono and diglycerides were then mixed. The fats were mixed for two minutes until homogenous. The fats were then mixed with 75% of the above cooled cooked mixture. The mixture was allowed to cool, forming a sugar-free composition.

The sugar-free composition was formed and allowed to set-up.

### Example 4

### Preparation of Sugar-Free Composition #4

A sugar-free base was prepared from 55% HSH and 20% lactitol, 0.50% kappa carrageenan and 20% water being added to a heating vessel. Following mixing, the mixture was heated to about 240° F. and a Brix of 87 to form a cooked mixture.

The cooked mixture was cooled to about 185-235° F. Next, 5.5% partially hydrogenated soy and cottonseed oil, 1.4% lecithin, and 4.5% mono and diglycerides were then mixed. The fats were mixed for two minutes until homogenous. The fats were then mixed with 75% of the above cooled cooked mixture. The mixture was allowed to cool, forming a sugar-free composition

The sugar-free composition was formed and allowed to set-up.

### Example 5

### Preparation of Sugar-Free Composition #5

A sugar-free base was prepared from 55% maltitol syrup and 20% lactitol, 0.50% iota carrageenan and 20% water being added to a heating vessel. Following mixing, the mixture was heated to about 240° F. and a Brix of 87 to form a cooked mixture.

The cooked mixture was cooled to about 185-235° F. Next, 8.0% partially hydrogenated soy and cottonseed oil, 1.4% lecithin, and 4.5% mono and diglycerides were then mixed. The fats were mixed for two minutes until homogenous. The fats were then mixed with 75% of the above cooled cooked mixture. The mixture was allowed to cool, forming a sugar-free composition.

The sugar-free composition was formed and allowed to set-up.

### Example 6

### Preparation of a Lemon-Lime Sugar-Free Composition Containing Several Fibers

A sugar-free base was prepared from 28% maltitol syrup and 11% lactitol, 4.0% partially hydrogenated soybean and cottonseed oil, 0.5% lecithin, 0.2% iota carageenan and 2.0% mono- and di-glycerides. Following mixing, the mixture was heated to about 240° F. and a Brix of 75 to form a cooked mixture.

The cooked mixture was cooled to about 185-235° F. Next, 6.0% glycerine, 4.0% flavors, and 0.23% sweeteners, 1.6% acidulents were then mixed. The cooled mixture was then mixed for two minutes until homogenous. The actives, including 6.80% psyllium husk, 20% inulin, and 10.80% cellulose, were then mixed with 50% of the above mixture. The mixture was allowed to cool, forming a fiber-based, sugar-free composition.

The fiber-based sugar-free composition was formed and allowed to set-up.

### Example 7

### Preparation of a Lemon-Lime Sugar-Free Composition Containing Several Fibers

A sugar-free base was prepared from 34% maltitol syrup and 12% lactitol, 6.0% partially hydrogenated soybean and cottonseed oil, 0.7% lecithin, 0.3% iota carageenan and 3.0% mono- and di-glycerides. Following mixing, the mixture was heated to about 240° F. and a Brix of 75 to form a cooked mixture.

The cooked mixture was cooled to about 185-235° F. Next, 6.0% glycerine, 4.0% flavors, and 0.23% sweeteners, 1.6% acidulents were then mixed. The cooled mixture was then mixed for two minutes until homogenous. The actives, including 6.80% psyllium husk, 20% inulin, and 10.80% cellulose, were then mixed with 50% of the above mixture. The mixture was allowed to cool, forming a fiber-based, sugar-free composition.

The fiber-based sugar-free composition was formed and allowed to set-up.

### Example 8

### Preparation of a Diabetic Sugar-Free Composition Containing Several Fibers

A protein-based, sugar-free base was prepared from 35% maltitol syrup, 15% lactitol and 0.08% carageenan being added to a heating vessel. Following mixing, the mixture was heated to about 240° F. and a Brix of 87 to form a cooked mixture.

The cooked mixture was cooled to about 185-235° F. Next, 4.95% partially hydrogenated soy and cottonseed oil, 0.77% lecithin, and 2.5% mono- and di-glycerides, 7.0% glycerine, 3.7% flavors and 1.83% sweeteners were then mixed. The ingredients were mixed for two minutes until homogenous. The ingredients were then mixed with 42% of the above cooled cooked mixture along with actives, including 23% specialty maltodextrin. The mixture was allowed to cool, forming a sugar-free composition.

The fiber-based, sugar-free composition was formed and allowed to set-up.

### Example 9

### Preparation of a Sugar-Free Composition Containing Several Fibers

A sugar-free base was prepared from 28% HSH and 10% lactitol, 4.0% partially hydrogenated soybean and cottonseed oil, 0.45% lecithin, 0.2% iota carrageenan and 2.2% mono- and di-glycerides being added to a heating vessel. Following mixing, the mixture was heated to about 240° F. and a Brix of 87 to form a cooked mixture.

The cooked mixture was cooled to about 185-235° F. Next, 6.0% glycerine, 4.0% flavors, and 0.22% sweeteners, 1.5% acidulents were then mixed. The ingredients were mixed for two minutes until homogenous. The ingredients were then mixed with 50% of the above cooled cooked mixture along with actives, including 6.80% psyllium husk, 20% inulin, and 10.80% cellulose. The mixture was allowed to cool, forming a fiber-based, sugar-free composition.

The fiber based sugar-free composition was formed and allowed to set-up.

### Example 10

### Preparation of a Sugar-Free Composition Containing Several Fibers

A sugar-free base was prepared from 34% HSH and 11% lactitol, 6.0% partially hydrogenated soybean and cottonseed oil, 0.7% lecithin, 0.3% iota carrageenan and 3.0% mono- and di-glycerides being added to a heating vessel. Following mixing, the mixture was heated to about 240° F. and a Brix of 87 to form a cooked mixture.

The cooked mixture was cooled to about 185-235° F. Next, 4.0% Grade A Non-fat dry milk, 6.0% glycerine, 4.0% flavors, and 0.22% sweeteners, 1.5% acidulents were then mixed. The ingredients were mixed for two minutes until homogenous. The ingredients were then mixed with 42% of the above cooled cooked mixture along with actives, including 6.80% psyllium husk, 20% inulin, and 10.80% cellulose. The mixture was allowed to cool, forming a fiber-based, sugar-free composition.

The fiber-based sugar-free composition was formed and allowed to set-up.

### Example 11

### Preparation of a Sugar-Free Composition Containing Several Fibers

A protein-based sugar-free base was prepared from 5.71% gelatin (150 Bloom), 20.40% glycerin (99.7%), 6.35% partially hydrogenated soy and cottonseed oil, 1.09% lecithin, 1.81% glyceryl monostearate by being added to a heating vessel. Following mixing, the mixture was heated to about 240° F. and a Brix of 87 to form a cooked mixture.

The cooked mixture was cooled to about 185-235° F. Next, 3.37% lactitol, 3.30% Grade A Non-fat dry milk, 11.01% glycerine, 4.10% flavors, and 1.30% acidulents were then mixed. The ingredients were mixed for two minutes until homogenous. The ingredients were then mixed with 76% of the above cooled cooked mixture along with the active, 41.30% psyllium husk. The mixture was allowed to cool, forming a protein-based, sugar-free composition.

The protein-based sugar-free composition was formed and allowed to set-up.

### Example 12

### Preparation of a Sugar-Free Composition Containing Several Fibers

A protein-based sugar-free base was prepared from 5.71% gelatin (150 Bloom), 20.40% glycerin (99.7%), 6.35% partially hydrogenated soy and cottonseed oil, 1.09% lecithin, 1.81% glyceryl monostearate by being added to a heating vessel. Following mixing, the mixture was heated to about 240° F. and a Brix of 87 to form a cooked mixture.

The cooked mixture was cooled to about 185-235° F. Next, 3.28% lactitol, 4.0% Grade A Non-fat dry milk, 11.01% glycerine, 4.10% flavors, and 1.30% acidulents were then mixed. The ingredients were mixed for two minutes until homogenous. The ingredients were then mixed with 75% of the above cooled cooked mixture along with actives, including 26.32% psyllium husk and 14.25% inulin. The mixture was allowed to cool, forming a protein-based, sugar-free composition.

The protein-based sugar-free composition was formed and allowed to set-up.

### Example 13

### Preparation of a Sugar-Free Composition Containing Fiber

A protein-based sugar-free base was prepared from 5.71% gelatin (150 Bloom), 20.40% glycerin (99.7%), 6.35% partially hydrogenated soy and cottonseed oil, 1.09% lecithin, 1.81% glyceryl monostearate by being added to a heating vessel. Following mixing, the mixture was heated to about 240° F. and a Brix of 87 to form a cooked mixture.

The cooked mixture was cooled to about 185-235° F. Next, 4.0% Grade A Non-fat dry milk, 10.01% glycerine, 4.10% flavors, and 1.60% acidulents were then mixed. The ingredients were mixed for two minutes until homogenous. The ingredients were then mixed with 78% of the above cooled cooked mixture along with actives, including 32.31% psyllium husk and 6.71% inulin. The mixture was allowed to cool, forming a protein-based, sugar-free composition.

The protein-based sugar-free composition was formed and allowed to set-up.

### Example 14

### Preparation of Protein-Based Sugar-Free Composition #6

A protein-based sugar-free base was prepared from 6.30% gelatin (150 Bloom), 20.40% glycerin (99.7%), 6.35% partially hydrogenated soy and cottonseed oil, 1.09% lecithin, 1.81% glyceryl monostearate by being added to a heating vessel. Following mixing, the mixture was heated to about 240° F. and a Brix of 87 to form a cooked mixture.

The cooked mixture was cooled to about 185-235° F. Next, 4.0% Grade A Non-fat dry milk, 10.01% glycerine, 4.10% flavors, and 1.60% acidulents were then mixed. The ingredients were mixed for two minutes until homogenous. The ingredients were then mixed with 72% of the above cooled cooked mixture along with the active, 32.31% psyllium husk. The mixture was allowed to cool, forming a protein-based, sugar-free composition.

The protein-based sugar-free composition was formed and allowed to set-up.

The inventive subject matter being thus described, it will be obvious that the same may be varied in many ways. Such variations are not to be regarded as a departure from the spirit and scope of the inventive subject matter, and all such modifications are intended to be included within the scope of the following claims.

What is claimed is:

1. A sugar-free composition comprising:
a) a mixture of at least two polyols present in an amount from about 15 to 80% by weight;
b) an emulsifier system present in an amount from about 1.0 to 30% by weight;
c) an active agent in an amount from about 0.1 to 70% by weight;
d) water in an amount from 0 to 15% by weight; and
e) optional components comprising colors, flavors and binders, wherein said ingredients in said sugar-free composition are present in an amount totaling 100%.

2. The sugar-free composition according to claim 1, wherein said polyols are selected from the group consisting of hydrogenated starch hydrolysate, isomalt, erythritol, polydextrose, maltitol, lactitol, glycerin, sorbitol, xylitol, mannitol and mixtures thereof.

3. The sugar-free composition according to claim 1 wherein said emulsifier system contains at least one emulsifier and at least one fat.

4. The sugar-free composition according to claim 3 where in said emulsifier is present in an amount from about 0.5 to 20.0% by weight of the final composition and said fat is present in an amount from about 1.0 to 10.0% by weight of the final composition.

5. The sugar-free composition according to claim 3, wherein said fat component is selected from the group consisting of chocolate, cocoa butter, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate, and mixtures thereof.

6. The sugar-free composition according to claim 3, wherein said emulsifier is selected from the group consisting of acetylated monoglycerides, glycerol esters, lecithin, de-oiled lecithin, enzyme-modified lecithins, purified lecithins, glycerol monostearate, polyglycerol esters, propylene glycol esters, sorbitan esters, polysorbate esters, sodium laurel sulfate, polyethylene glycols, sorbitol mono-, di- and tri-stearates and mixtures thereof.

7. The sugar-free composition according to claim 1, wherein said active agent is selected from the group consisting of dietary fiber, stannol esters, therapeutically active substances, vitamins, minerals, antacids, cough and cold medications, analgesics, cardiovasular medications, anti-smoking, psycho-therapeutics, antibiotics, and mixtures thereof.

8. The sugar-free composition according to claim 1, further comprising a viscosity improvement agent present in an amount from about 0.1 to 10% by weight of the final composition.

9. The sugar-free composition according to claim 7, wherein said viscosity improvement agent is selected from the group consisting of locust bean gum, guar gum, hydrolyzed guar gum, carrageenan, starches, gum arabic, gelatin, agar, alginate, pectin and mixtures thereof.

10. The sugar-free composition according to claim 1, further comprising a bioadhesive agent.

11. The sugar-free composition according to claim 10, wherein said bioadhesive agent is selected from the group consisting of hydroxypropylmethyl cellulose, ethyl cellulose, acrylic esters, polyvinyl acetates, alcohols and gums.

12. The sugar-free composition according to claim 1, wherein said mixture of at least two polyols is present in an amount of about 20 to 50% by weight.

13. The sugar-free composition according to claim 1, wherein said active agent is an amount of about 1.0 to 50% by weight.

14. The sugar-free composition according to claim 1, wherein said active agent is at least one fiber.

15. The sugar-free composition according to claim 14, wherein said fiber is selected from the group consisting of noncellulosic polysaccharides, pectin, gums, algal polysaccharides, specialty maltodextrins, cellulose, hemicellulose, fructo-oligo saccharides, psyllium, lignin, mucilages and mixtures thereof.

16. A protein-based, sugar-free composition comprising a mixture of:
a) a mixture of at least one protein and at least one polyol present in an amount from about 20 to 99% by weight;
b) an emulsifying system present in an amount from about 1.0 to 30% by weight;
c) an active agent in an amount from about 0.1 to 70% by weight; and
d) water in an amount from 0 to 10% by weight.

17. The protein-based, sugar-free composition according to claim 16, wherein said polyols are selected from the group consisting of hydrogenated starch hydrolysate, isomalt, erythritol, polydextrose, maltitol, lactitol, glycerin, sorbitol, xylitol, mannitol and mixtures thereof.

18. The protein-based, sugar-free composition according to claim 16, wherein said emulsifier system contains at least one emulsifier and at least one fat.

19. The protein-based, sugar-free composition according to claim 16, wherein said emulsifier is present in an amount from about 0.5 to 20.0% by weight of the final composition and said fat is present in an amount from about 1.0 to 10.0% by weight of the final composition.

20. The protein-based, sugar-free composition according to claim 16, wherein said fat component is selected from the group consisting of chocolate, cocoa butter, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate, and mixtures thereof.

21. The protein-based, sugar-free composition according to claim 16, wherein said emulsifier is selected from the group consisting of acetylated monoglycerides, glycerol esters, lecithin, de-oiled lecithin, enzyme-modified lecithins, purified lecithins, glycerol monostearate, polyglycerol esters, propylene glycol esters, sorbitan esters, polysorbate esters, sodium laurel sulfate, polyethylene glycols, sorbitol mono-, di- and tri-stearates and mixtures thereof.

22. The protein-based, sugar-free composition according to claim 16, wherein said active agent is selected from the group consisting of dietary fiber, stannol esters, therapeutically active substances, vitamins, minerals, antacids, cough and cold medications, analgesics, cardiovasular medications, anti-smoking, psycho-therapeutics, antibiotics, and mixtures thereof.

23. The protein-based, sugar-free composition according to claim 16, further comprising a viscosity improvement agent present in an amount from about 0.1 to 10% by weight of the final composition.

24. The protein-based, sugar-free composition according to claim 16, wherein said viscosity improvement agent is selected from the group consisting of locust bean gum, carrageenan, starches, gum arabic, gelatin, agar, alginate, pectin and mixtures thereof.

25. The protein-based, sugar-free composition according to claim 16, further comprising a bioadhesive agent.

26. The protein-based, sugar-free composition according to claim 16, wherein said bioadhesive agent is selected from the group consisting of hydroxypropylmethyl cellulose, ethyl cellulose, acrylic esters, polyvinyl acetates, alcohols and gums.

27. The protein-based, sugar-free composition of claim 16, wherein said protein is selected from the group consisting of gelatin from cows, pigs, and fish and mixtures thereof.

28. The sugar-free composition of claim 1, further comprising a liquid or powdered lime flavor in an amount of 3.5-4.0% by weight.

29. The sugar-free composition of claim 1, further comprising a liquid or powdered tropical flavor in an amount of 3.6-4.1% by weight.

30. The sugar-free composition of claim 1, further comprising cool-mint, menthol flavor encapsulation, eucalyptus oil, peppermint liquid and peppermint flavor encapsulation wherein an oral hygiene formulation is formed.

31. The protein-based, sugar-free composition according to claim 16, wherein said one protein is present in an amount of about 1 to 35% by weight.

32. The protein-based, sugar-free composition according to claim 16, wherein said one polyol is present in an amount of about 1.5 to 70% by weight.

33. The protein-based, sugar-free composition according to claim 16, wherein said polyol is present in an amount of about 1.5-2.0 times the amount of said protein present.

34. The protein-based composition according to claim 16, wherein said active agent is at least one fiber.

35. The protein-based composition according to claim 34, wherein said fiber is selected from the group consisting of noncellulosic polysaccharides, pectin, gums, algal polysaccharides, specialty maltodextrins, cellulose, hemicellulose, fructo-oligo saccharides, psyllium, lignin, mucilages and mixtures thereof.

36. A method of preparing a sugar-free composition comprising the steps of:
a) mixing from about 15 to 80% by weight of at least two polyols with about 0 to 15% by weight water;
b) heating said mixture to about 200-260° F. for about 5 to 10 minutes;
c) cooling said mixture to about 175-240° F.;
d) adding to said mixture about 1.0 to 30% by weight an emulsifier system to form a further mixture;
e) cooling said further mixture to about 100-110° F.;
f) adding to said cooled further mixture about 0.1 to 70% by weight of an active ingredient to form said sugar-free composition.

37. The method according to claim 36, wherein said heating in step b) takes place under a vacuum.

38. An oral hygiene composition comprising:
a) a mixture of at least two polyols present in an amount from about 50 to 99% by weight;
b) an emulsifier system present in an amount from about 1.0 to 30% by weight;
c) an active ingredient in an amount from about 0.0 to 20% by weight;
d) a bioadhesive agent for improving oral hygiene; and
e) water in an amount from 0 to 25% by weight.

39. A weight reduction or cholesterol reducing composition comprising:
a) a mixture of at least two polyols present in an amount from about 50 to 99% by weight;
b) an emulsifier system present in an amount from about 1.0 to 30% by weight;
c) an active agent in an amount from about 0.0 to 20% by weight, said active agent being selected from the group consisting of dietary fibers, hydroxycitric acid, vitamins, minerals, and mixtures thereof; and
d) water in an amount from 0 to 15% by weight.
